(19) United States
(12) Patent Application Publication
(10) Pub. No.: US 2025/0354921 A1
(43) Pub. Date: Nov. 20, 2025

(54) OIL DIAGNOSIS METHOD AND OIL DIAGNOSIS SYSTEM

(71) Applicant: Hitachi, Ltd., Tokyo (JP)

(72) Inventor: Kyoko KOJIMA, Tokyo (JP)

(73) Assignee: Hitachi, Ltd., Tokyo (JP)

(21) Appl. No.: 18/874,027

(22) PCT Filed: May 12, 2023
(86) PCT No.: PCT/JP2023/017936

§ 371 Date: Dec. 11, 2024

(30) Foreign Application Priority Data
Jul. 7, 2022 (JP) 2022-110079

(51) Int. Cl. G01N 21/31; G01N 33/28

(52) U.S. Cl. CPC G01N 21/3103; G01N 33/2888

(57) ABSTRACT

An object of the invention is to reduce the effects of a change in light source intensity and a decrease in sensitivity of the optical sensor to accurately determine an oil state when color change of an oil (measurement target) is measured using an optical sensor to detect a change in oil properties. One preferred aspect of the invention is a method for diagnosing a state of an oil containing additives, the oil absorbing light in a wavelength range of 400 to 800 nm, where the state of the oil is determined by a ratio of the transmittances at two different wavelengths with different light transmittances.

### TECHNICAL FIELD

The present invention relates to an oil diagnostic technique. In particular, the invention relates to maintenance of large machinery using industrial oil such as lubricating oil, insulating oil, and processing oil, specifically relates to a technique to monitor the machinery by diagnosing the remaining life of the oil by measuring color change of the oil, such as the lubricating oil, with use.

### BACKGROUND ART

Diagnosing properties of the lubricating oil used in bearings, gears, and other rotating components is an important technique for maintenance of large rotating machinery. Examples of the large rotating machinery include, for example, a speed-increasing gear of a wind turbine generator, an air compressor, a ship, and a power generation turbine.

In addition to the lubricating oil, the insulating oil is used for electrical insulation in a transformer, etc., and it is also important to diagnose properties of the insulating oil. In addition, the processing oil, etc. is used for machining. There are various types of processing oils for different applications, such as a cutting oil, a press oil, a heat treatment oil, and a rust preventive oil. In this Description and others, the industrial oil such as the lubricating oil, the insulating oil, and the processing oil may be collectively referred to as oil.

Depending on the purpose of use, there are various types of lubricating oil, such as engine oil, turbine oil, hydraulic oil, bearing oil, sliding surface oil, gear oil, compressor oil, and cutting oil. Various additives are blended into a base oil (oil to be a base) to ensure that each type of lubricating oil meets the required performance. Other types of oil may be blended with additives to have required properties.

Recent machine state monitoring often involves a strategy to minimize life cycle cost of a machine. The large machinery such as a power generation turbine uses a large amount of lubricating oil and is stopped to replace the lubricating oil, which disadvantageously leads to power generation loss, production stoppage, and the like, and requires costs for purchasing and delivering a new oil, oil replacement, and waste oil disposal, and thus the lubricating oil is desirably used as long as possible. Refrigerant liquids, etc. for electric vehicles and data centers are also subjected to oil diagnosis for replacement or hardware repairs. The insulating oil for a transformer is also monitored for color, etc.

Recently, from the viewpoint of carbon neutrality, demand for fuel will decrease in the future due to electrification of automobiles and other vehicles that use a large amount of petroleum-based fuel, but there are often no alternatives for industrial oils, and there is a demand to minimize the amount of oil used by extending the oil replacement cycle, etc. This is because reducing oil consumption reduces carbon dioxide emissions. However, overlooking oil deterioration and contamination can lead to machine failure.

In diagnosis of lubricating oil properties, “deterioration” and “contamination” are respectively defined to be distinguished from each other. Broadly speaking, two diagnoses need to be performed for (1) oxidative deterioration of the lubricating oil over time, and (2) contamination by external contaminants such as water, dust, and wear particles.

The oxidative deterioration of the lubricating oil (1) includes deterioration due to oxidation of the base oil and deterioration due to additive consumption. The oxidative deterioration of the lubricating oil causes a decrease in wear resistance, changes in viscosity and viscosity index, a decrease in a rust prevention property, and a decrease in an anticorrosion property. As a result, wear and material fatigue of the speed-increasing gear may be accelerated. While the lubricating oil is desirably used as long as possible, if any abnormal deterioration or contamination is found, oil replacement and device inspection should be promptly performed.

As for transmittance changes at the three wavelengths of RGB (red, green, blue) in visible light, as described in Patent Literature 1, there is a method diagnosing timing of lubricating-oil replacement and signs of machine abnormalities based on a relative change in transmittance from that of new oil. The Patent Literature 1 describes that the diagnosis is performed based on a change in B value, which shows the largest change in transmittance due to oil deterioration among the RGB values.

### CITATION LIST

### Patent Literature

Patent Literature 1: Japanese Unexamined Patent Application Publication No. 2019-078718

### SUMMARY OF INVENTION

### Technical Problem

The oil deterioration and contamination are each observed as a decrease in ΔE value. On the other hand, when long-term oil monitoring is assumed, for example, during long-term continuous measurement in a high-temperature environment, light intensity of a light source such as a light-emitting diode (LED) may decrease. Furthermore, sensitivity of a semiconductor sensor to detect RGB light may decrease.

When light source intensity decreases, a decrease in ΔE value is observed, and whether the decrease in ΔE value is due to a change in oil state or a decrease in light intensity of the light source may not be apparent only by checking the sensor output value.

Decrease in sensitivity of the RGB sensor also results in decrease in ΔE value, which is less likely to be distinguished from the decrease in ΔE value due to oil deterioration.

In the method using RGB three-wavelength detection, even in the method of diagnosis based on a change in B value, which is the largest change in transmittance due to oil deterioration, when the B value changes, it is difficult to determine whether the value change is caused by oil deterioration, a decrease in light source intensity, or a decrease in sensitivity of the RGB sensor.

In a high-performance benchtop analyzer such as a spectrophotometer, a method using a reference light, called a double beam method, can be used to eliminate effects of fluctuations in light source intensity and in detection sensitivity, leading to accurate transmittance measurement. However, the double-beam structure has been less likely to be used for a small optical sensor.

An object of the invention, which has been found during the investigation to solve the above problems, is to reduce the effects of a change in light source intensity and a decrease in sensitivity of the optical sensor to accurately determine an oil state when color change of an oil (measurement target) is measured using an optical sensor to detect a change in oil properties.

### Solution to Problem

One preferred aspect of the invention is a method for diagnosing a state of an oil containing additives, the oil absorbing light in a wavelength range of 400 to 800 nm, where the state of the oil is determined by a ratio of the transmittances at two different wavelengths with different light transmittances.

Another preferred aspect of the invention is an oil diagnosis system including a light source and a light receiving element having sensitivities to at least two different source is wavelengths, where a visible light from the light transmitted through an oil, and the visible light transmitted through the oil is detected by the light receiving element to acquire two pieces of chromaticity information corresponding to the two different wavelengths, and the oil state is diagnosed based on a ratio of the two pieces of chromaticity information.

### Advantageous Effects of Invention

According to the invention, when color change of an oil (measurement target) is measured using an optical sensor to detect a change in properties of the oil, effects of a change in light source intensity and a decrease in sensitivity of the optical sensor can be reduced, leading to accurate determination of a state of the oil. Other problems, configurations, and effects will be clarified by the following description of some embodiments.

### BRIEF DESCRIPTION OF DRAWINGS

FIG. 1 is a graph showing color change of a gear oil with use.

FIG. 2 is a graph showing spectral sensitivity characteristics of an RGB color sensor.

FIG. 3 is a graph showing color change with mixing-in of water.

FIG. 4 is a graph showing changes in various indices for gear oil deterioration.

FIG. 5 is a table showing color change of a gear oil with use.

FIG. 6 is a graph showing an example of a calibration curve obtained from a correlation between a B/R value and concentration of an extreme-pressure agent in a gear oil.

FIG. 7 is a graph showing an example of a calibration curve obtained from a correlation between an R/B value and concentration of the extreme-pressure agent in the gear oil.

FIG. 8 is a graph showing an example of a calibration curve obtained from a correlation between a total acid number of a gear oil and B/R.

FIG. 9 is a graph showing results of measurement, performed every month, of a gear oil with an optical sensor.

FIG. 10 is a graph showing results of measurement of a gear oil with an optical sensor.

FIG. 11 is a table showing results of measurement of color change of a gear oil with use, the measurement being performed every month using an optical sensor capable of measuring the color change as RGB color coordinates.

FIG. 12 is a graph showing changes in various indices for gas turbine oil deterioration.

FIG. 13 is a graph showing changes in various indices for engine oil deterioration.

FIG. 14 is a schematic view of a monitoring system of a lubricating oil for a wind turbine generator.

FIG. 15 is a conceptual view of a rotating component with a lubricating-oil sensor.

FIG. 16 is a flowchart showing a lubricating-oil diagnostic process.

FIG. 17 is a graph showing the concept of an acquisition result of lubricating-oil chromaticity data (B/R values) stored on a time-series basis.

FIG. 18 is a graph showing a relationship between TPPT concentration and elapsed time.

FIG. 19 is a table showing transmittance measurement results and transmittance ratios between two wavelengths.

FIG. 20 is a graph showing temporal changes in the transmittance ratios between the two wavelengths.

FIG. 21 is a graph showing a correlation between the transmittance ratios between the two wavelengths and the degree of contamination determined by a mass method.

### DESCRIPTION OF EMBODIMENTS

Some embodiments will be described in detail with reference to the drawings. However, the invention should not be construed as being limited to the description of the following embodiments. It will be readily understood by those skilled in the art that the specific configuration of each embodiment can be modified or altered within the scope without departing from the idea or the gist of the invention.

In the configurations of the embodiments described below, the same reference numerals are commonly used between different drawings for portions that are identical or have similar functions, and duplicated description may be omitted.

When there are multiple elements having the same or similar functions, they may be described using the same reference numerals with different subscripts. However, when there is no need to distinguish between the multiple elements, the elements may be described using the reference numerals with the subscripts omitted.

In this Description and others, the terms “first,” “second,” “third,” and the like are merely used to identify components, and do not necessarily limit the number, order, or content of each component. The numbers used to identify components are used on a context-by-context basis, and a number used in one context does not necessarily indicate the same configuration in another context. A component identified by a certain number is not precluded from having the function of a component identified by another number.

The position, size, shape, range, etc. of each component shown in the drawings etc. may not represent the actual position, size, shape, range, etc. in order to facilitate understanding of the invention. The invention is therefore not necessarily limited to the position, size, shape, range, etc. shown in the drawings, etc.

Any of the publications, patents and patent applications cited herein forms part of the explanation of this Description without modification.

Components expressed in the singular herein shall include the plural unless the context otherwise clearly indicates.

The method for monitoring machinery using color change of an oil such as a lubricating oil according to the embodiments is characterized in that diagnosis of deterioration and contamination of the oil using a visible optical sensor is performed with a ratio of values indicating transmittances at any two different wavelengths as an index, enabling accurate observation of oil deterioration. If (transmittance=1−absorptance) is true in principle, then transmittance and absorptance have the same significance in the embodiments.

The lubricating oil includes various types of oils, such as engine oil, turbine oil, hydraulic oil, bearing oil, sliding surface oil, gear oil, compressor oil, and cutting oil. The lubricating oil is configured of base oil and additives. The additives include an antioxidant, a rust inhibitor, an antifoaming agent, a viscosity index improver, an oiliness improver, an extreme pressure additive, a detergent dispersant, a pour point depressant, and an emulsifier. Oils used for purposes other than lubrication include transformer oil. For many oils, color change with use is an index of determination of replacement time or machine abnormality.

The lubricating oil includes a base oil and additives, where the base oil includes a mineral oil made from petroleum, a high-performance synthetic oil, and a bio-oil made from a plant. The synthetic oil is highly pure, extremely chemically stable, and is less likely to be deteriorated. Color change of the lubricating oil including the synthetic oil is therefore often caused by consumption of the additives. On the other hand, the mineral oil and the bio-oil each have low purity or a somewhat unstable chemical ester structure, and thus may cause coloring of the base oil when used. The base oil may also be colored due to consumption of the additives.

For color change of the oil, specifically, while new oil is colorless or pale yellow, it becomes yellow, orange, reddish brown, and then blackish brown with the passage of days of use.

### Color Change Due to Deterioration

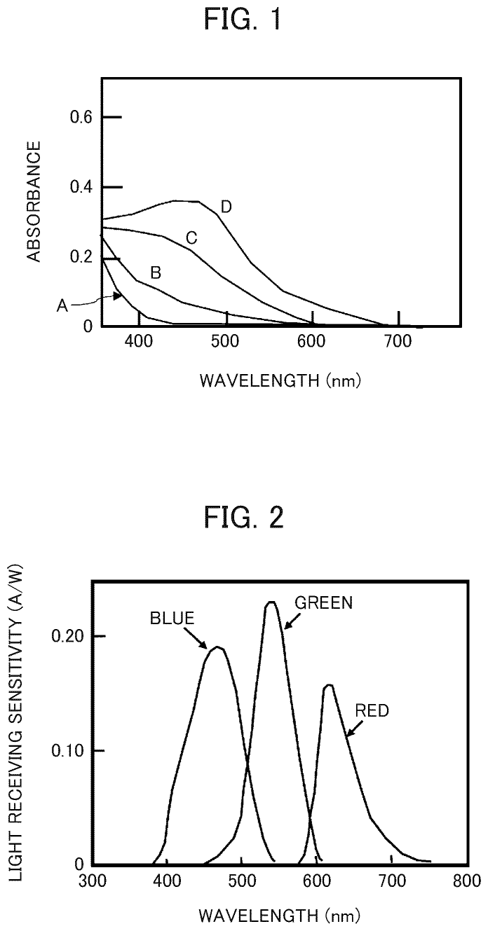

FIG. 1 shows color change of a gear oil with use. The vertical axis indicates absorbance of the oil at a wavelength indicated on the horizontal axis. A shows visible absorbance of a new oil, B shows that of the oil used for two months, C shows that of the oil used for six months, and D shows that of the oil used for one year.

The apparent color of the new oil A is slightly yellowish but almost colorless and transparent, B is pale yellow, C is orange, and D is reddish brown. Similar color change also occurs in engine oil, engine oil, turbine oil, hydraulic oil, bearing oil, sliding surface oil, compressor oil, cutting oil, rolling oil, and insulating oil.

FIG. 2 shows spectral sensitivity characteristics of an RGB color sensor including a Si photodiode array. A three-channel (RGB) photodiode sensitive to blue (wavelength 460 nm), green (wavelength 540 nm), and red (wavelength 620 nm) is used for the Si photodiode array. This color sensor has a spectral sensitivity characteristic close to a luminosity factor, and can be used to express an oil color as color coordinates.

### Color Change Due to Contamination

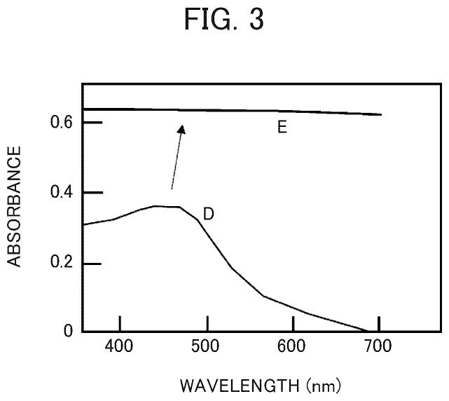

FIG. 3 illustrates a relationship between wavelengths and absorbance as in FIG. 1, showing an example (E) where a gear oil D, which has been used for one year, is contaminated by water. While D has been reddish brown and transparent, E is cloudy brown due to 1 wt % water contamination, resulting in an increase in absorbance at all wavelengths in the visible range, i.e., a decrease in transmittance.

The absorbance and transmittance are defined as follows. When light passes through a sample with a certain optical path length,

Absorbance=log(Iᵢ/I₀)

Transmittance (%)=(I₀/Iᵢ)×100

where Iᵢ represents incident light intensity, and I₀ represents transmitted light intensity. As is clear, absorbance and transmittance are mutually convertible.

When the color change (light transmittance) of the gear oil as shown in FIG. 1 is measured by the RGB color sensor of FIG. 2, the R (Red) value shows a small amount of change, the B (Blue) value shows a larger amount of change than R and G, and the G (Green) value shows an amount of change, which is larger than R but smaller than B.

A color of a new gear oil is measured by an RGB color sensor, and color coordinates of the new oil are set to (255, 255, 255). The color coordinates of the new oil can also be set to, for example, (100, 100, 100), or may be set to values such as (100, 97, 80). A color of a used oil or a deteriorated oil can be measured by the same method as that for the new oil.

The color coordinates of a gear oil that has been used for one year are measured in the same method as the method of measuring the new oil with a sensor having the same performance as the sensor used for measuring the new oil. Deterioration of the gear oil is evaluated by a ratio of the B value, with the maximum amount of change due to deterioration, to the R value, with the minimum amount of change due to deterioration, i.e., the value of (B/R). In the evaluation method, the reciprocal (R/B) value may be used for evaluation. The ratios of B to G, i.e., the (B/G) value and the (G/B) value, and the ratios of R to G, i.e., the (R/G) value and the (G/R) value, may also be used.

At this time, if light intensity of a light source has decreased to 90% of that at new oil measurement, since each of the RGB values becomes 90% of that at the new oil measurement even if there is no deterioration or contamination of the oil, whether such a decrease in the value is due to deterioration or contamination of the oil or a change in light source intensity cannot be determined by the diagnosis based on the RGB values and the ΔE value. If two values are selected from the RGB values, and a ratio of the two values, such as (B/R), is used for evaluation, the effect of the change in light source intensity can be reduced.

Although sensor performance has been assumed to be unchanged in the above, if sensitivity of the sensor has decreased to 90% of that at the new oil measurement, since each of the RGB values becomes 90% of that at the new oil measurement even if there is no oil deterioration or contamination, whether such a decrease in the value is due to deterioration or contamination of the oil or a change in light source intensity cannot be determined by the diagnosis based on the RGB values and the ΔE value. If two values are selected from the RGB values, and a ratio of the two values, such as (B/R), is used for evaluation, the effect of the change in sensitivity of the sensor can be reduced.

Even if both the light intensity of the light source and the sensor sensitivity are changed, effects of the changes can be reduced by using the ratio of the two values for evaluation.

Other than RGB Sensor

In addition to detection with the RGB sensor, in case of measurement a camera capable of with multispectral simultaneously measuring 10 to 20 wavelengths in the visible range or a spectrophotometer with excellent wavelength resolution, any two wavelengths can be selected for diagnosis using the ratio between transmittances or absorbances at the respective wavelengths. At this time, if the two wavelengths are selected from 400 to 500 nm for one and from 600 to 700 nm for the other, the color change due to oil deterioration can be diagnosed with high accuracy.

### Contamination Detection During Continuous Measurement

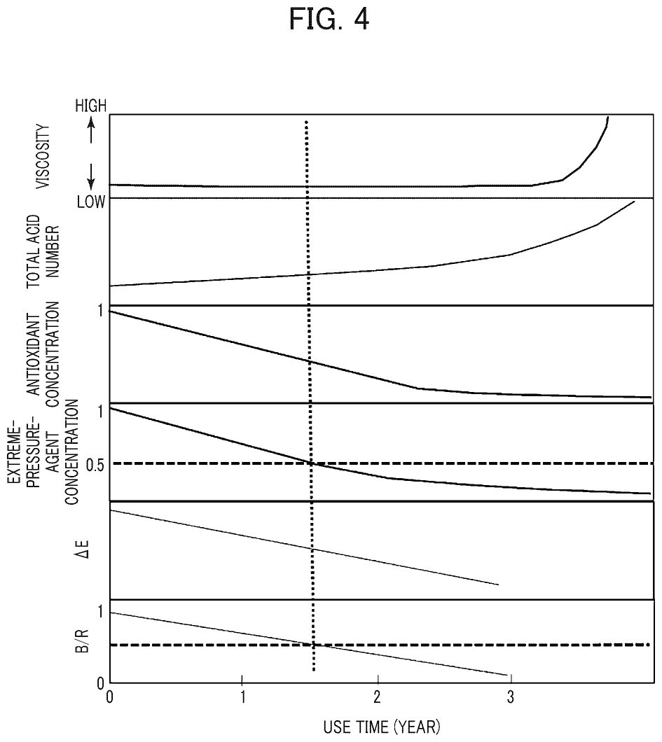

In case of measuring a change in oil deterioration over time using as an index the ratio of two values selected from the RGB values, If mixing-in of water or generation of a large amount of wear particles occurs during the measurement, for example, while the ratio (B/R) continues to decrease during proceeding of the deterioration, if contamination occurs, the light transmittance decreases regardless of wavelengths, and thus (B/R) approaches 1. FIG. 4 shows an example of contamination detection in this manner. As described above, diagnosis using the ratio is also effective in diagnosing oil contamination.

### First Embodiment

The following is an example of diagnosis of a gear oil for a wind-turbine speed increasing gear.

FIG. 4 shows changes in viscosity, total acid number, and concentrations of an antioxidant and an extreme-pressure agent, and changes in ΔE and (B/R) measured by an optical sensor to show deterioration of the gear oil, containing the antioxidant and the extreme-pressure agent, for the wind-turbine speed increasing gear.

The apparent color of the lubricating oil changes from colorless to yellow, then to brown, that is, becomes darker as deterioration progresses. Replacement of the gear oil is determined when the extreme-pressure agent concentration reaches 50% of the initial value. The timing at which the extreme-pressure agent concentration becomes 50% of the initial value can be determined by the timing at which B/R becomes equal to or less than a predetermined threshold.

Although the above determination was made based on the extreme-pressure agent concentration, the determination could also be made using the total acid number, viscosity, antioxidant concentration, etc.

### Example where Light Source Intensity is Unchanged

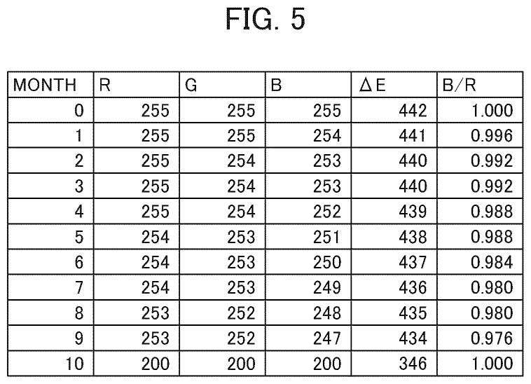

FIG. 5 shows results of measurement of color change of the gear oil with use, the measurement being performed every month using an optical sensor capable of measuring the color change as RGB color coordinates. After the measurement was completed, it was confirmed that when such data were acquired, there was no decrease in light source intensity and in sensitivity of the optical sensor.

The color coordinates of a new oil (R, G, B) were set to (255, 255, 255) expressed in 8 bits. The color coordinates may be expressed in percent, (100, 100, 100), or the like, in addition to 8-bit expression, and the coordinate system may be a color system other than the RGB color system.
ΔE was defined as follows.

$$Δ E = {( {R}^{2} + {G}^{2} + {B}^{2} )}^{⋀} ( 1 / 2 )$$

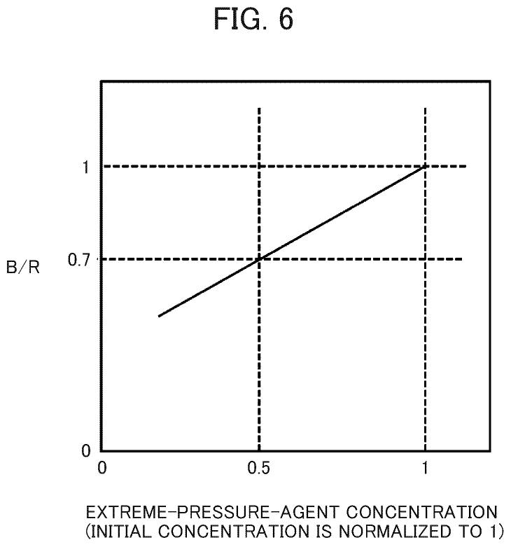

Decreases in light source intensity and in sensitivity of the optical sensor may be ignored in the measurements of the calibration curves in the following FIGS. 6 to 8.

FIG. 6 shows an example of a calibration curve obtained from a correlation between a value of B/R and concentration of the extreme-pressure agent in the gear oil. The initial concentration of the extreme-pressure agent in a new oil was normalized to 1, and the values of the new oil measured by the optical sensor were set to (255, 255, 255). The concentration of the extreme-pressure agent in the gear oil was determined quantitatively by high performance liquid chromatography. It had been determined that the oil was replaced when the extreme-pressure agent concentration reached 50% of the initial concentration. When the extreme-pressure agent concentration reached 50% of the initial value, B/R was 0.7.

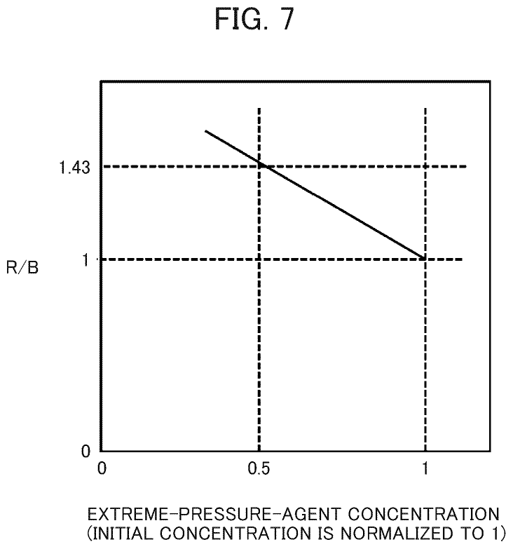

FIG. 7 shows an example of a calibration curve obtained from a correlation between a value of R/B and concentration of the extreme-pressure agent in the gear oil. The initial concentration of the extreme-pressure agent in the new oil was normalized to 1, and the values of the new oil measured at that time by the optical sensor were set to (100, 100, 100). The concentration of the extreme-pressure agent in the gear oil was determined quantitatively by high performance liquid chromatography. It had been determined that the oil was replaced when the extreme-pressure agent concentration reached 50% of the initial concentration. When the extreme-pressure agent concentration reached 50% of the initial value, R/B was 1.43.

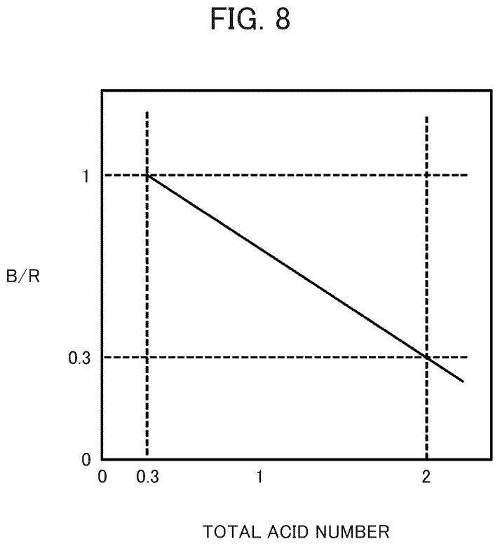

FIG. 8 shows an example of a calibration curve obtained from a correlation between the total acid number of the gear oil and B/R as a color index. This calibration curve was prepared in advance using a new oil and a deteriorated oil. The total acid number was measured by a titration method using an indicator. The values of the new oil measured by the optical sensor were set to (255, 255, 255). The total acid number of the new oil is 0.3, and this gear oil is recommended to be replaced when the total acid number exceeds 2. The calibration curve revealed that when the total acid number was 2, B/R was 0.3.

Such calibration curves can be created from a correlation of the physical property parameters of the gear oil with output of the optical sensor.

Any two of RGB values can be selected as output values of the optical sensor, and a calibration curve can be created using the ratio between the output values. In addition to R/B and B/R, R/G, G/R, B/G, and G/B could also be used.

### Example of Detection of Water Contamination

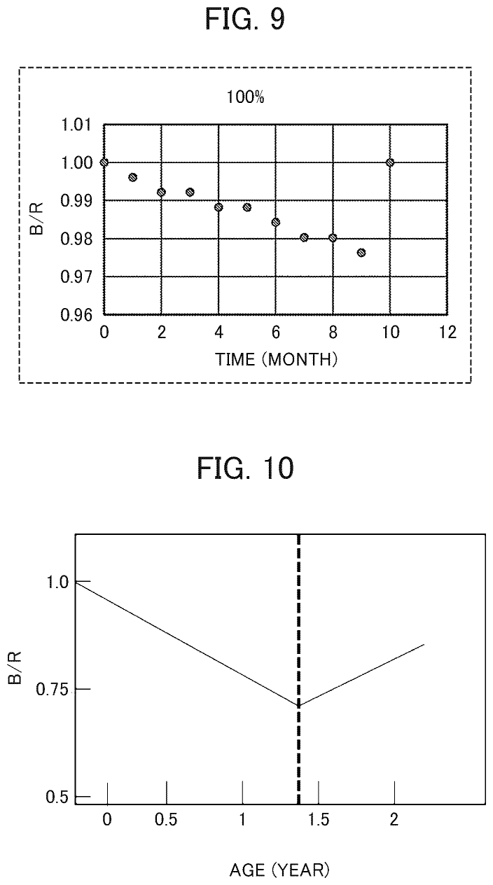

FIG. 9 shows results of measurement of a gear oil for a speed-increasing gear of an onshore wind turbine, the measurement being performed every month using an optical sensor. In a period between the 9th and 10th months, 2% by weight of water was mixed in and the gear oil became cloudy. The B/R value continued to decrease until the 9th month, and then became 1 in the 10th month. Thus, 100 ml of the gear oil was sampled and analyzed, and mixing-in of water was confirmed.

### Example of Detection of Contamination by Abrasion Powder

FIG. 10 shows results of continuous measurement, by an optical sensor, of the gear oil for the speed-increasing gear of the onshore wind turbine. After 1.4 years, the B/R value changed from decrease to increase. After a lapse of two years, 100 ml of the gear oil was sampled and analyzed, and mixing-in of abrasion powder was confirmed. Although contamination due to the abrasion powder, sand, etc. may gradually progress over a long period of time, the contamination could be detected by the method of this embodiment.

### Comparative Example

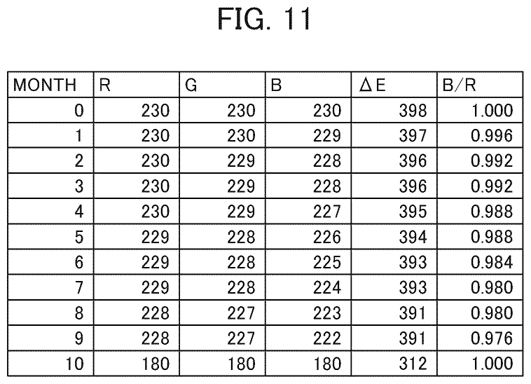

FIG. 11 shows results of measurement of color change with use of a gear oil for a speed-increasing gear of a wind turbine, the measurement being performed every month using an optical sensor capable of measuring the change as RGB color coordinates. The measured samples were the same as those in FIG. 5. The color coordinates (R, G, B) of a new oil were set to (255, 255, 255).

The optical sensor was left for one month after setting of the new oil value, and when the data shown in FIG. 11 was acquired, the light source intensity of the optical sensor had decreased by 10%. As a result, the RGB values each decreased by 10%, and the ΔE value correspondingly decreased. On the other hand, the same values as those of the measurement results in FIG. 5 were obtained for the B/R values. This means that taking a ratio of pieces of color information makes it possible to ignore the effect of changes in light source intensity of the optical sensor.

### Second Embodiment

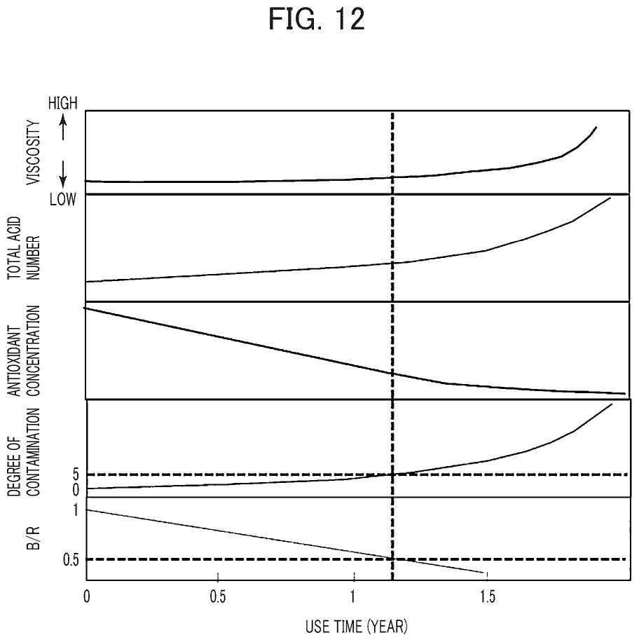

FIG. 12 shows an example of diagnosis of a gas turbine oil for a large ship. FIG. 12 specifically shows changes with use in viscosity, total acid number, antioxidant concentration, degree of contamination, and B/R value measured by an optical sensor, of the gas turbine oil. The color coordinates (R, G, B) of a new oil were set to (255, 255, 255).

The degree of contamination is a value determined by the mass method defined by the JIS B 9931 standard. An increase in degree of contamination means an increase in organic insoluble matter due to oxidation of the gas turbine oil. There is a correlation between the degree of contamination and the B/R value, and thus a calibration curve can be beforehand created to monitor the increase in the degree of contamination by measuring the B/R value. In the example of FIG. 12, the timing of the contamination degree 5 can be detected when the B/R value becomes approximately 1.2 or less.

The degree of contamination, based on the mass method, has a strong correlation with generation of sludge or varnish in the lubricating oil, and thus generation of the sludge or the varnish can be predicted by monitoring the degree of contamination.

Although monitoring of the degree of contamination has been exemplarily described in the above, viscosity, total acid number, and antioxidant concentration can also be monitored in the same way using the B/R value.

### Third Embodiment

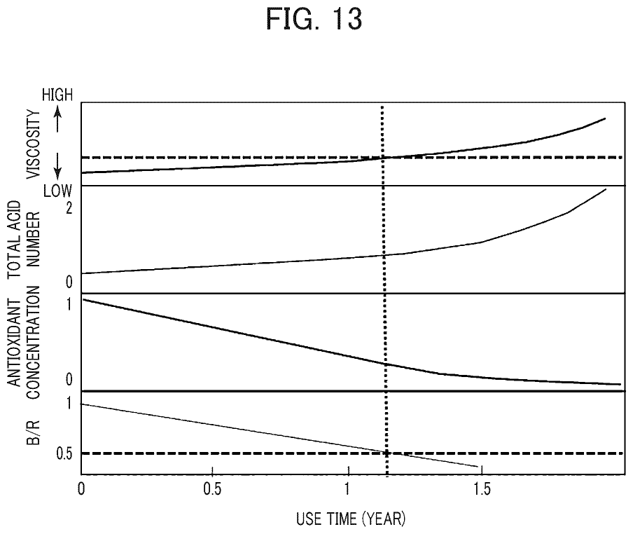

FIG. 13 shows an example of diagnosis of an engine oil for a gasoline vehicle. FIG. 13 specifically shows the changes with use in viscosity, total acid number, antioxidant concentration, and B/R of an engine oil including a mineral oil as a base oil. The color coordinates (R, G, B) of a new oil were set to (255, 255, 255).

The viscosity is kinematic viscosity (unit: cp) at 40° C., and the antioxidant concentration is relative concentration (the antioxidant concentration of new oil was set to 1) determined by Fourier transform infrared spectroscopy (FT-IR). The viscosity of this engine oil increases with use, and the viscosity could be monitored using a calibration curve created from the correlation between the viscosity and the B/R value.

The total acid number and antioxidant concentration can also be monitored using the B/R value.

### Fourth Embodiment

In this embodiment, the above monitoring method is applied to a system and a method for monitoring a lubricating oil for a wind turbine generator. This embodiment includes a system for monitoring the lubricating oil supplied to a mechanical drive unit of the wind turbine generator. The system includes an input device, a processing device, a storage device, and an output device. The storage device stores additive concentration data in which concentrations of additives in the lubricating oil are stored on a time-series basis, and the processing device estimates time, at which the additive concentration in the lubricating oil, which is determined from chromaticity characteristics of the lubricating oil, reaches a predetermined threshold, based on optical sensor data to measure the lubricating-oil chromaticity that allows the additive concentration in the lubricating oil to be quantitated.

Further, this embodiment includes a method for monitoring the lubricating oil for the wind turbine generator with an optical lubricating-oil sensor, using a server including a processing device, a storage device, an input device, and an output device. In this method, the following steps are performed: A first step of acquiring chromaticity data of the lubricating oil for the wind turbine generator, a second step of measuring concentration of additives in a sample, a third step of storing, as additive concentration data, the measured additive concentrations in the storage device on a time-series basis, and a fourth step, in which the processing device processes the additive concentration data to estimate the time at which the additive concentration reaches a predetermined threshold.

### (1. Overall System Configuration)

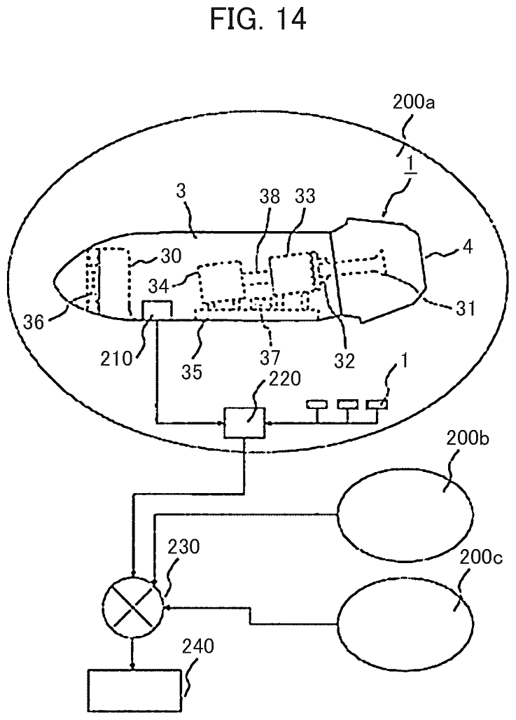

FIG. 14 is a schematic view of a monitoring system of a lubricating oil for a wind turbine generator having a lubricating-oil supply system. Inside a nacelle 3 of a wind turbine generator 1, there is a spindle 31, a speed-increasing gear 33, a generator 34, and undepicted bearings for yaw, pitch, and the like, to each of which a lubricating oil is supplied from an oil tank 37. The monitoring system further includes a typical configuration of the wind turbine generator, including a hub 4, a nacelle bulkhead 30, a shrink disk 32, a main frame 35, a radiator 36, and a coupling 38.

As shown in FIG. 14, there are a plurality of wind turbine generators 1, which are typically installed on the same site and collectively referred to as farm 200a or the like. Each wind turbine generator 1 has various sensors (not shown) installed in the supply system of the lubricating oil, and sensor signals reflecting states of the lubricating oil are collected in a server 210 in the nacelle 3.

A sensor signal obtained from the servers 210 of the wind turbine generator 1 is sent to an aggregation server 220 disposed in each firm. Data from the aggregation server 220 is sent via a network 230 to a central server 240. Data are also sent to the central server 240 from other farms 200b and 200c. The central server 240 can send an instruction to each wind turbine generator 1 via the aggregation server 220 and the server 210. In this way, the system of this embodiment enables remote monitoring of the oil.

### (2. Sensor Placement)

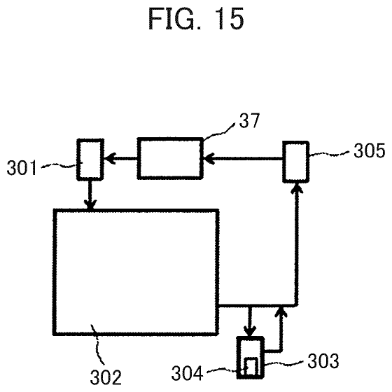

FIG. 15 is a conceptual view of a rotating component with a lubricating oil sensor. A lubricating oil is supplied to a rotating component 302 from a lubricating-oil supply device 301 such as a pump. The lubricating oil supply device 301 is connected to an oil tank 37 and receives the lubricating oil. The rotating component 302 general part that may be subjected to mechanical contact, for example, but not limited to, the speed-increasing gear 33.

The optical sensor 304 is placed in a lubricating-oil flow path, etc. to detect the state of the lubricating oil. In this embodiment, a measurement section 303 is provided in a flow path (near the end of the lubricating-oil flow path) which branches off from the lubricating-oil flow path connected to a lubricating-oil drain outlet of the rotating component 302, and the lubricating oil is partly introduced into the measurement section 303. The optical sensor 304 is installed in the measurement section 303. The measurement section 303 is thus not provided in the main flow path of the lubricating oil in order to adjust the flow rate of the lubricating oil in the measurement unit 303 to a flow rate suitable for detecting the state of the lubricating oil. The lubricating oil discharged from the rotating component 302 returns to the oil tank 37 through the filter 305. The filter 305 is however not essential. The optical sensor 304 measures the color coordinates of the lubricating oil. The state of the lubricating oil can be evaluated based on temporal changes in color coordinates of the lubricating oil.

In this embodiment, the optical sensor 304 includes an optical sensor including a visible light source and a light receiving element. The optical sensor acquires chromaticity information (R, G, B values) of the lubricating oil. The optical sensor 304 transmits the visible light from the visible light source through the oil, and acquires chromaticity data by detecting the visible light transmitted through the oil with the light receiving element having sensitivities to R, G, and B, respectively. The acquired chromaticity data reflects absorptance or transmittance of the oil for each wavelength of light. A ratio of the two wavelengths is determined from the acquired chromaticity data, and amount of remaining additives in the lubricating oil is determined using a calibration curve beforehand determined from a correlation between a ratio of the two wavelengths and the amount of the remaining additives to diagnose a deterioration level and a remaining life.

The lubricating oil deteriorates in quality with use and no longer performs its original function. It is therefore necessary to perform maintenance such as replacement depending on deterioration state of quality. To know timing of such maintenance, it is useful for efficiency of maintenance management to enable remote monitoring of data collectable by the optical sensor 304 installed on-site. The data collected by the optical sensor 304 is collected, for example, in the server 210 in the nacelle 3, and then sent to the central server 240, which aggregates data from multiple farms, via the aggregation server 220 that aggregates data within the farm 200.

However, for analysis that requires special equipment for measurement, such as liquid chromatography (LC) measurement, FT-IR measurement, and nuclear magnetic resonance (NMR) measurement, it is necessary to collect a sample of the lubricating oil as appropriate and conduct the analysis using separately installed equipment. Desirably, the results measured by such LC, FT-IR, and NMR measurements are also separately stored as data in the central server 240, and the data are aggregated and taken into consideration to grasp properties of the lubricating oil.

The data to be aggregated may include not only data on the lubricating oil, but also data indicating an operating state of the wind turbine generator. For example, the data may include a wind turbine output value (the larger the value, the greater the deterioration rate of the lubricating oil), actual operating time (the longer the time is, the greater the deterioration rate of the lubricating oil), machine temperature (the higher the temperature, the greater the deterioration rate of the lubricating oil), shaft rotation speed (the faster the speed, the greater the deterioration rate of the lubricating oil), and the like. Such data can be collected from sensors with known configurations installed at various locations in the wind turbine generator and from control signals for devices.

### (3. Lubricating Oil Diagnosis Flow)

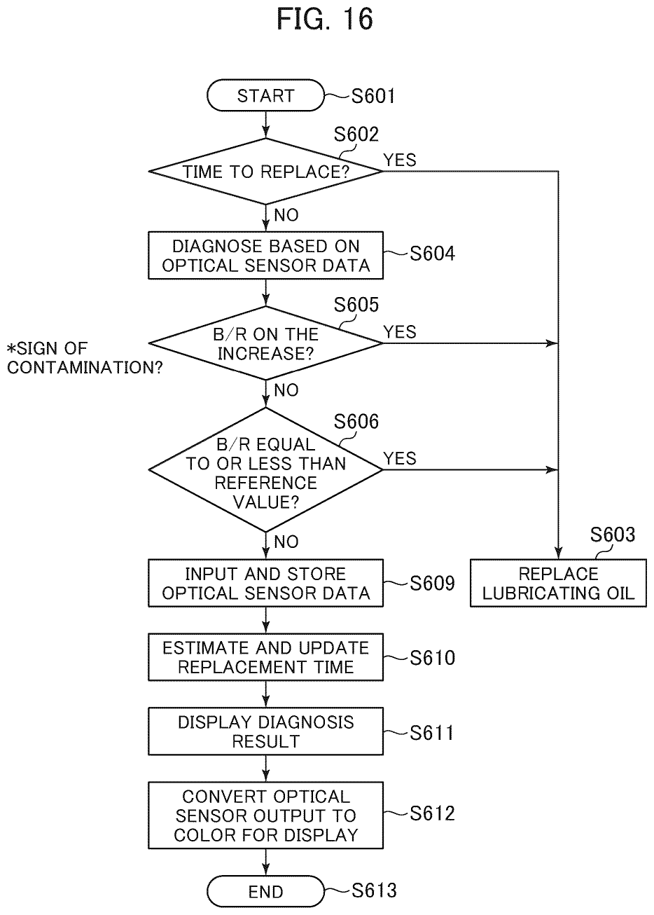

FIG. 16 is a flowchart showing lubricating-oil diagnostic processing according to this embodiment. The processing shown in FIG. 16 is performed under the control of any one of the servers, i.e., the server 210, the aggregation server 220, and the central server 240, in FIG. 14. In the following example, the central server 240 performs the processing. Functions such as computation and control are implemented by cooperation of the server with other hardware to perform specified processing through execution by a processor of software stored in the storage device of the server. The functions equivalent to those configured in software can also be implemented in hardware such as field programmable gate array (FPGA) and application specific integrated circuit (ASIC).

When the central server 240 performs control, since there are multiple wind turbine generators 1 to be controlled, the following processing is performed for each wind turbine generator. The processing is basically repetitive processing, and the start timing is set by a timer or the like. For example, the processing starts at 0:00 every day (S601). Alternatively, the central server 240 can perform the control at any timing according to an instruction from an operator.

In step S602, the central server 240 checks whether it is time to replace the lubricating oil. The initial value of the replacement time is calculated physically by using the Arrhenius reaction rate, the assumption that the lubricating oil is operating at a design temperature, enabling initial setting of the remaining life. Such replacement time can be updated later in step S610 based on actual measurement data.

If it is time to replace the lubricating oil, the lubricating oil is replaced in step S603. Since the lubricating-oil is usually replaced by an operator, the central server 240 provides display and notification to instruct the operator on the replacement time and the replacement object.

If it is not time to replace the lubricating oil, the central server 240 performs a diagnosis based on the sensor data in step S604. Temperature, oil pressure, concentration of particles in the lubricating oil, etc. can be used as the sensor data, in addition to the chromaticity information of the lubricating oil obtained by the optical sensor. The data collected by the optical sensors 304 are sent to the central server 240, which evaluates the characteristics of the lubricating oil, for example, by comparing a parameter obtained from the sensor with a predefined threshold.

If there is any abnormality in results of the diagnoses in steps S605 and S606, the lubricating oil is replaced in step S603. If there is no abnormality, step S609 is performed. In step S605, for example, if the B/R value based on the R, G, B values of the optical sensor changes from decrease to increase, it is determined that there is a contamination abnormality.

In S606, using the correlation between the additive concentration and B/R as shown in FIG. 6, it is determined that when the additive concentration determined from the B/R value measured by the optical sensor falls below a predetermined threshold, there is an abnormality in the additive deterioration level. It is also possible to determine that there is an abnormality in the additive deterioration level when B/R becomes smaller than a predetermined threshold, without determining the additive concentration from the B/R value.

In step S609, the chromaticity measurement data and the like are input into the central server 240, and such data are stored on a time-series basis.

From the viewpoint of preventive and planned maintenance of the wind turbine generator, it is desirable to perform a predictive diagnosis of deterioration of the lubricating oil based on transition of concentration of additives contained in the lubricating oil before any abnormality is determined to exist.

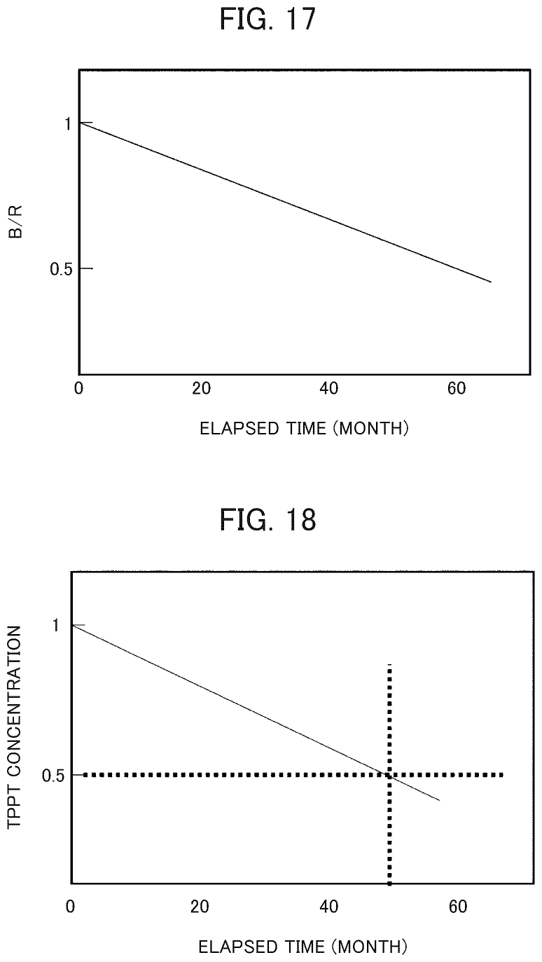

FIG. 17 is a graph showing the concept of an acquisition result of lubricating-oil chromaticity data (B/R values) stored on a time-series basis. The horizontal axis indicates time (month), and the vertical axis indicates B/R. For example, assuming that B/R is monitored at a fixed point, the chromaticity data up to 60 months are plotted. A significant relationship is observed between the elapsed time and B/R, for example, B/R decreases linearly with time. As for the data, the values of (R, G, B) may be stored as data to calculate the B/R values, or calculated B/R values may be stored as data.

The concentration of the additives such as the extreme-pressure agent in the lubricating oil can be determined from the chromaticity data ((R, G, B) values) using the correlation between B/R and the additive concentration as shown in FIG. 6. The consumption rate of the additives therefore can be calculated from the chromaticity measurement results stored on a time-series basis. Assume that if the additive concentration becomes approximately half that of a new product, performance of the lubricating oil falls below the acceptable range. Such a threshold can be determined experimentally.

In this example, in step S610, the threshold of the additive concentration is set to 0.5, and the replacement time is estimated to be the time when the concentration, which is estimated from the additive concentration measurement results stored on a time-series basis, reaches 0.5. Various known methods may be used for the estimation. If actual measurement values have been obtained, known methods of extrapolating data can be used on the assumption that the concentration decreases monotonically. When the concentration transitions in a complicated manner, a known method such as function fitting (curve fitting) can be used.

In this embodiment, the time-series chromaticity data measured by the optical sensor, i.e., B/R, are stored, and the deterioration level of the lubricating oil is estimated based on the data.

The results of the replacement time estimation in step S610 can be displayed as the lubricating-oil diagnostic results (step S611). FIG. 18 shows a display example of the results of step S610.

In the example of FIG. 18, the additive is an extreme-pressure agent, triphenyl phosphorothionate (TPPT). It was estimated that B/R would reach the threshold of 0.5 after approximately fifty months. Since the TPPT concentration will reach 50 after fifty months, new replacement time should be set to a time before that (for example, half a month before). One cycle of processing is completed in step S613, and determination processing is performed according to the new replacement time in step S602 of the subsequent cycle.

For example, after S611, the chromaticity data measured by the optical sensor can be converted into color and displayed on the display screen of the diagnostic results of the lubricating oil. The deterioration state of the lubricating oil is thus shown in color on the display screen, allowing an operator to visually recognize the deterioration state of the lubricating oil. This helps, for example, an operator to roughly grasp a deteriorated state of the lubricating oil when the operator visually observes the state of the lubricating oil in the field.

As described above, according to this embodiment, the life of the lubricating oil can be detected early without being affected by variation in performance of the optical sensor, by using the correlation between the ratio of two of the RGB values measured by the optical sensor and the additive concentration created in advance based on the results of the composition analysis. It is therefore possible to prevent any abnormality in the wind turbine generator by maintenance such as appropriate replacement of the lubricating oil. It is also possible to optimize a replacement cycle of the lubricating oil. The additive concentration can be measured by a simple method, and if the optical sensor is installed inside the nacelle, it is possible to remotely monitor deterioration of the additives in the lubricating oil online.

Although this embodiment has been described with the method and system for monitoring with the optical sensor installed in the lubricating oil for the rotating component, the same diagnosis can also be performed by sampling a lubricating oil in the rotating component during inspection or the like, and measuring the sampled oil with an optical sensor outside the rotating component.

### Fifth Embodiment

An optical sensor capable of measuring continuous spectra in the visible range (400 to 800 nm) was used to diagnose oxidation deterioration of a gas turbine oil. In this diagnosis, the wavelength resolution was 10 nm, and transmittance (%) of the gas turbine oil was measured each time the wavelength increases by 10 nm from 400 nm. White LED was used as the light source, and the measurement optical path length was 10 mm.

The gas turbine was operated continuously, and 50 ml of oil was sampled every month to measure light transmittance. Tλ represents the light transmittance (%) at a wavelength λ. Values of light transmittance ratios between two wavelengths, T₄₃₀/T₄₅₀, T₄₃₀/T₅₅₀, and T₄₃₀/T₇₀₀, were determined from the measurement results.

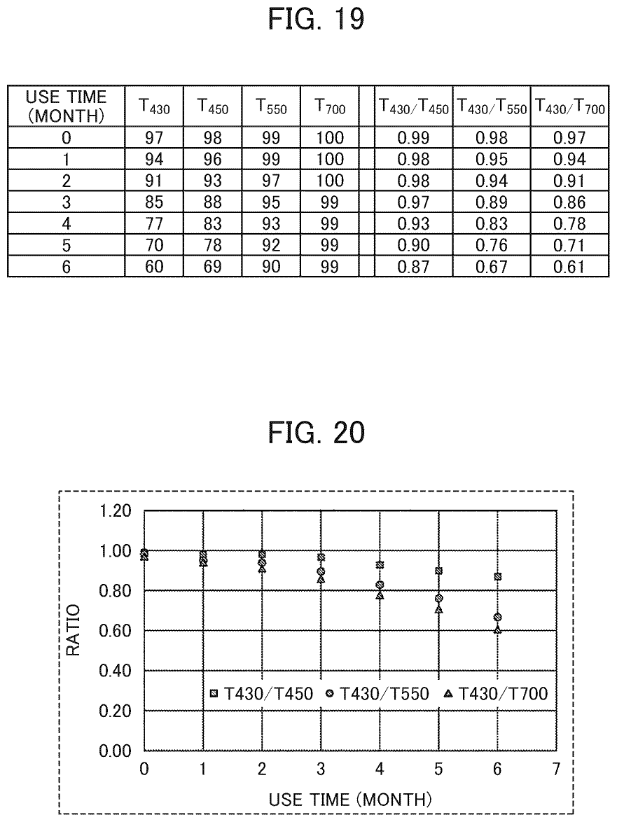

FIG. 19 is a table showing the transmittance measurement results and the transmittance ratios between the two wavelengths.

FIG. 20 is a graph showing temporal changes in the transmittance ratios between the two wavelengths.

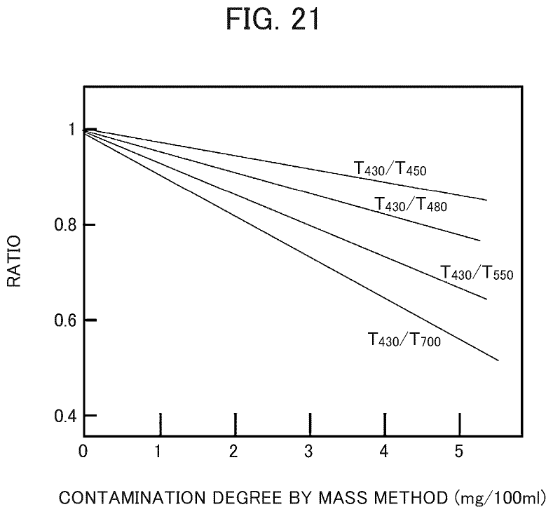

FIG. 21 is a graph showing a correlation between the transmittance ratios between the two wavelengths and the degree of contamination determined by a mass method.

As described above, a calibration curve was created from the relationship between the ratio of transmittances at any two wavelengths in the visible range and the degree of contamination, and the results of the optical sensor measurements could be used to estimate the degree of contamination of the lubricating oil.

As seen from the results, it was possible to monitor the degree of contamination with high sensitivity by selecting two wavelengths 50 nm or more apart from each other, such as 430 nm and 480 nm, rather than selecting two wavelengths close to each other, such as 430 nm and 450 nm. Furthermore, the degree of contamination could be monitored with even higher sensitivity by selecting two wavelengths 120 nm or more, preferably 270 nm or more, apart from each other, such as 430 nm and 550 nm or 430 nm and 700 nm.

As described in the embodiments, since R and B of the RGB color sensor configured of an easily available Si photodiode array are away by approximately 160 nm from each other, it is possible to monitor the characteristics of the oil by simply taking the signal ratio without any additional hardware while reducing effects of variation in output of the light source or the sensor.

Similarly, viscosity, total acid number, or the like can be monitored using an optical sensor by creating a calibration curve based on the correlation with a ratio of transmittances at any two wavelengths.

For the ratio, transmittance, absorbance, analog output values (voltage value, current value) of the detector of the optical sensor, or the like can also be used for the diagnosis in the same way.

For the oil type, although gear oil and gas turbine oil have been exemplified, another type of lubricating oil, processing oil, insulating oil, etc., can also be diagnosed in the same way.

According to the above embodiments, for the optical sensor of the RGB three-wavelength output type, it is possible to reduce effects of changes on the sensor side, such as changes in light source intensity and a decrease in sensitivity of the RGB detection sensor, on color change of oil (a liquid to be measured). It is therefore possible to accurately grasp the remaining life of the lubricating oil by the optical sensor.

According to the above embodiments, efficient oil maintenance and management can be achieved, resulting in less energy consumption, reduced carbon emissions, prevention of global warming, and contribution to realization of a sustainable society.

### LIST OF REFERENCE SIGNS

1: wind turbine generator
3: nacelle
4: hub

1. A method for diagnosing a state of an oil containing an additive, the oil absorbing light in a wavelength range of 400 to 800 nm, wherein the state of the oil is determined by a ratio of transmittances at two different wavelengths with different light transmittances.

2. The method according to claim 1,
wherein the two different wavelengths are two wavelengths 50 nm or more apart from each other.

3. The method according to claim 2,
wherein the two different wavelengths are two wavelengths 120 nm or more apart from each other.

4. The method according to claim 3,
wherein the two different wavelengths are two wavelengths 270 nm or more apart from each other.

5. The method according to claim 1,
wherein transmittances at RGB three wavelengths are determined, and
the state of the oil is determined by a ratio of transmittances at two wavelengths selected from the RGB three wavelengths.

6. The method according to claim 5,
wherein the wavelength R and the wavelength B are selected as the two wavelengths.

7. The method according to claim 1,
wherein the state of the oil is determined using a calibration curve obtained from a correlation between the transmittance ratio and viscosity, total acid number, additive concentration, and/or a degree of contamination of the oil.

8. The method according to claim 1,
wherein the state of the oil is determined by monitoring the transmittance ratio as time-series data.

9. The method according to claim 8,
wherein if trend of the transmittance ratio is reversed between increase and decrease in the time series data, a foreign substance is determined to be mixed in the oil.

10. An oil diagnosis system, comprising a light source and a light receiving element having sensitivities to at least two different wavelengths,
wherein a visible light from the light source is transmitted through the oil, and the visible light transmitted through the oil is detected by the light receiving element to acquire two pieces of chromaticity information corresponding to the two different wavelengths, and
a state of the oil is diagnosed based on a ratio of the two pieces of chromaticity information.

11. The oil diagnosis system according to claim 10,
wherein the two different wavelengths are two wavelengths 50 nm or more apart from each other.

12. The oil diagnosis system according to claim 11,
wherein the two different wavelengths are two wavelengths 120 nm or more apart from each other.

13. The oil diagnosis system according to claim 12,
wherein the two different wavelengths are two wavelengths 270 nm or more apart from each other.

14. The oil diagnosis system according to claim 10,
wherein the state of the oil is diagnosed based on the ratio of the two pieces of chromaticity information acquired on a time-series basis.

15. The oil diagnosis system according to claim 10,
wherein the light source is a visible light source, and the light receiving element is a RGB color sensor to acquire pieces of chromaticity information of R, G, and B, and the state of the oil is diagnosed based on a ratio of pieces of chromaticity information of R and B.
